(19) United States
(12) Patent Application Publication
(10) Pub. No.: US 2024/0232574 A9
(43) Pub. Date: Jul. 11, 2024

(54) METHOD AND APPARATUS FOR PREDICTING RECIPE PROPERTY REFLECTING SIMILARITY BETWEEN CHEMICAL MATERIALS

(71) Applicant: Pusan National University Industry-University Cooperation Foundation, Busan (KR)

(72) Inventors: HYE RIM BAE, Busan (KR); han byeol Park, Busan (KR); MINGYU PARK, Busan (KR); DOHEE KIM, Busan (KR); KI HUN KIM, Busan (KR)

(21) Appl. No.: 18/475,206

(22) Filed: Sep. 27, 2023

(30) Foreign Application Priority Data
Oct. 19, 2022 (KR) 10-2022-0134746

(51) Int. Cl. G06N 3/04

(52) U.S. Cl. CPC G06N 3/04

(57) ABSTRACT

A method and apparatus for predicting a recipe property reflecting similarity between chemical materials are provided. The method of predicting a recipe property reflecting similarity between chemical materials, the method includes substituting a plurality of input materials with vector data, respectively, through material embedding and generating recipe data including pieces of vector data selected by considering a correlation between materials, and inputting the recipe data to an artificial neural network (ANN) prediction model and deriving a property prediction result on the recipe data from the ANN prediction model.

### CROSS-REFERENCE TO RELATED APPLICATION

This application claims the benefit of Korean Patent Application No. 10-2022-0134746 filed on Oct. 19, 2022, in the Korean Intellectual Property Office, the entire disclosure of which is incorporated herein by reference for all purposes.

### BACKGROUND

### 1. Field of the Invention

The present disclosure relates to a method and apparatus for predicting a recipe property reflecting similarity between chemical materials, more particularly, predicting a recipe property by reflecting similarity between rubber materials to predict an accurate property of a rubber material recipe.

Particularly, the present disclosure provides a method and device for predicting a recipe property reflecting similarity between chemical materials for predicting an accurate property of a rubber recipe using input data by defining similarity from a type and an injection amount of a material, and expressing the material as a vector based on the similarity.

### 2. Description of the Related Art

A property of a rubber product may have a range to be satisfied that varies depending on the type of a product, and various methods of predicting the property are proposed.

It is known that a property of a product is difficult to predict due to the nonlinear characteristics of various materials. However, recently, with the development of artificial neural network (ANN) technology, a prediction problem due to nonlinear characteristics, such as rubber property prediction, has been solved.

Examples of prior inventions are a method of predicting a property using a characteristic (e.g., a tensile strength) of natural rubber as an input variable, and a method of predicting a tensile reaction using an input amount and a type of a material having a small number of processes.

A conventional method of predicting a property uses a characteristic of rubber as an input variable and a conventional method of predicting a tensile reaction uses an input amount and an input of key materials as input variables.

However, a rubber recipe may exponentially increase whenever the number of cases for inputting or an available input material is added, and due to this, research using various input materials has limitations.

For a problem that needs to consider various input materials, material data, which is categorical data, may need to be used as input data.

Embedding may refer to a process of substituting categorical data with numerical data.

As a traditional embedding method, one-hot encoding may be provided.

However, when converting data into a vector using one-hot encoding, similarity between vectors may not be considered and there is a spatial limitation that a dimension needs to be added whenever expressing one vector.

To overcome such limitations, embedding has been used using a method such as Word2Vec.

The method of using Word2Vec is based on a natural language similarity hypothesis.

The natural language similarity hypothesis is based on a distribution hypothesis that a target word is similar to a word existing near the target word.

However, if the similarity of a rubber material is assumed based on the natural language similarity hypothesis, a contradiction that defines the similarity between completely irrelevant rubber materials may occur.

Therefore, there is a demand for a new framework for defining appropriate similarity for a new domain, which is a rubber material, converting a similar rubber material into a vector, and predicting a property using the converted material vector.

### SUMMARY

An embodiment of the present invention provides an apparatus and method of predicting a recipe property reflecting similarity between chemical materials to define similarity between rubber materials that may not be considered in a conventional embedding method, and to reconstruct input data by expressing a material as a vector based on the similarity.

In addition, an embodiment of the present invention provides a method of predicting an accurate property of a rubber recipe using reconstructed data as input data to an artificial neural network.

According to one embodiment of the present invention, a method of predicting a recipe property reflecting similarity between chemical materials, the method includes substituting a plurality of input materials with vector data, respectively, through material embedding and generating recipe data including pieces of vector data selected by considering a correlation between materials, and inputting the recipe data to an artificial neural network (ANN) prediction model and deriving a property prediction result on the recipe data from the ANN prediction model.

In addition, according to one embodiment of the present invention, an apparatus for predicting a recipe property reflecting similarity between chemical materials, the apparatus includes a generation unit configured to substitute a plurality of input materials with pieces of vector data through material embedding, respectively, and generate recipe data including pieces of vector data selected by considering a correlation between materials, and a processing unit configured to input the recipe data to an ANN prediction model and derive a property prediction result for the recipe data from the ANN prediction model.

Additional aspects of example embodiments will be set forth in part in the description which follows and, in part, will be apparent from the description or may be learned by practice of the disclosure.

According to one embodiment of the present invention, an apparatus and method of predicting a recipe property reflecting similarity between chemical materials are provided to define similarity between rubber materials that may not be considered in a conventional embedding method and to reconstruct input data by expressing a material as a vector based on the similarity.

In addition, according to the present invention, an accurate property of a rubber recipe is predicted using reconstructed data as input data to an artificial neural network.

According to a method proposed by the present invention, as a result of a performance comparison experiment with existing proposed models for rubber material data, it is proved that the performance is improved by up to 37% on average.

### BRIEF DESCRIPTION OF THE DRAWINGS

These and/or other aspects, features, and advantages of the invention will become apparent and more readily appreciated from the following description of example embodiments, taken in conjunction with the accompanying drawings of which:

FIG. 1 is a block diagram illustrating a configuration of an apparatus for predicting a recipe property reflecting similarity between chemical materials according to one embodiment of the present invention;

FIG. 2 is a diagram illustrating a whole process of material embedding according to the present invention;

FIG. 3 is a diagram illustrating a property prediction model based on an artificial neural network according to the present invention; and

FIG. 4 is a flowchart illustrating a method of predicting a recipe property reflecting similarity between chemical materials according to one embodiment of the present invention.

### DETAILED DESCRIPTION

Hereinafter, example embodiments will be described in detail with reference to the accompanying drawings. However, various alterations and modifications may be made to the embodiments. Here, the embodiments are not meant to be limited by the descriptions of the present disclosure. The embodiments should be understood to include all changes, equivalents, and replacements within the idea and the technical scope of the disclosure.

The terminology used herein is for the purpose of describing particular example embodiments only and is not to be limiting of the example embodiments. The singular forms “a”, “an”, and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms including technical and scientific terms used herein have the same meaning as commonly understood by one of ordinary skill in the art to which example embodiments belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

When describing the embodiments with reference to the accompanying drawings, like reference numerals refer to like constituent elements and a repeated description related thereto will be omitted. In the description of embodiments, detailed description of well-known related structures or functions will be omitted when it is deemed that such description will cause ambiguous interpretation of the present disclosure.

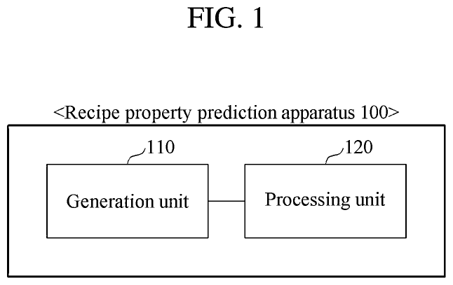

FIG. 1 is a block diagram illustrating a configuration of an apparatus for predicting a recipe property reflecting similarity between chemical materials according to one embodiment of the present invention.

Referring to FIG. 1, according to one embodiment of the present invention, a device for predicting a recipe property reflecting similarity between chemical materials 100 (hereinafter, referred to as the “recipe property prediction apparatus”) may include a generation unit 110 and a processor 120.

First of all, the generation unit 110 may substitute a plurality of input materials with pieces of vector data, respectively, through material embedding and may generate recipe data including vector data selected by considering a correlation between materials. That is, the generation unit 110 may convert each of input materials, which are substances, into vector data, which is a numerical value, and may generate recipe data by selecting and including pieces of vector data having mutual correlation.

In this case, the recipe data may refer to data related to a method of determining a type, an input amount, and the like of a material, which is a raw material for producing a product. The recipe data may function as source data that informs of a property of a product produced by the recipe data based on training thereafter.

The generation unit 110 may generate recipe data related to product production using vector data of materials having correlations therebetween.

In substituting each input material into vector data, the generation unit 110 may divide and cluster the plurality of materials by type. That is, the generation unit 110 may group and divide input materials into clusters by type and category. For example, the generation unit 110 may divide input materials into clusters, which are rubber R, carbon C, and oil O.

In addition, the generation unit 110 may substitute divided clusters with vector data using a random variable X.

The random variable may be a function that determines real values as a distribution by an intuitively determined probability with respect to an unknown sample that is a subject of single observation, and may refer to a function X that assigns a real number x(=X(w)) to an element w in a sample space.

In addition, the processing unit may calculate a mean and a variance (Var) for the substituted vector data. That is, the processing unit 120 may calculate Mean:E(X₁) and Var:E(X₁²)−E(X₁)² by substituting vector data for a mean calculation equation and variance calculation equation.

In substituting with vector data, the generation unit 110 may substitute a material, which is predefined but is not an input to ???, with vector data having a determined vector value. That is, the generation unit 110 may substitute data, which is not actually an input to ???, with vector data of a zero vector.

In the above-described example in which rubber R, carbon C, and oil O are input, when a material that is predefined but is not an input is silicon, the generation unit 110 may substitute the silicon with vector data having a value of “0”.

After calculating the mean and variance, the generation unit 110 may show a distribution status by performing K-means clustering using the calculated mean and variance.

K-means clustering may be an algorithm for clustering given data into k clusters and may operate in a way that minimizes a variance of a distance difference of each cluster. K-means clustering may iteratively update a center point using mean coordinates of points allocated to each cluster and may show a distribution status on a virtual plane.

The generation unit 110 may dispose k centroids on a virtual plane through K-means clustering, may form a cluster by allocating each piece of data to the nearest centroid, may calculate a new centroid based on the allocated cluster, and may show the distribution status by repeating the above-described processes.

In addition, the generation unit 110 may implement the shown distribution status in graph data. That is, the generation unit 110 may implement graph data by setting a cluster to be a vertex in the shown distribution status and connecting the clusters with a line.

In addition, the generation unit 110 may perform random walk sampling on the implemented graph data and may generate a sequence of similar materials. That is, the generation unit 110 may connect multiple pieces of graph data and may generate a sequence of similar materials by arranging materials based on similarity.

Thereafter, the generation unit 110 may construct a matrix with respect to a material type and material name with the sequence of similar materials. That is, the generation unit 110 may express multiple pieces of the connected graph data by a matrix of a material type and material name.

In addition, the generation unit 110 may reconstruct the matrix by performing Word2Vec embedding. That is, the generation unit 110 may reconstruct the constructed matrix by converting the matrix into a vector through Word2Vec embedding.

Thereafter, the generation unit 110 may generate the recipe data by selecting significant data in the reconstructed matrix based on the correlation between the materials. That is, the generation unit 110 may select significant data having high mutual correlation in the reconstructed matrix and may generate the recipe data by including the selected significant data.

In addition, the processing unit 120 may input the recipe data into an artificial neural network (ANN) prediction model and may derive a property prediction result on the recipe data from the ANN prediction model. That is, for recipe data generated by considering the mutual correlation between materials, the processing unit 120 may predict a property of a product to be produced based on the recipe data through training.

The processing unit 120 may apply the recipe data to a prediction model, may form an ANN hidden layer using the recipe data through the prediction model, and may derive the property prediction result from a material property and material attention score using the ANN hidden layer.

That is, the processing unit 120 may receive an output of a material property and a material attention score as a training result of the prediction model related to the recipe data.

According to one embodiment, the processing unit 120 may decrease a training processing amount in the prediction model by decreasing a dimension of the ANN hidden layer.

For this, the processing unit 120 may derive an attention score for each ANN hidden layer, may concatenate the ANN hidden layers by considering the attention scores, and may derive the property prediction result for the ANN hidden layer of which a dimension decreases through flattening.

That is, the processing unit 120 may sequentially concatenate and flatten ANN hidden layers to which high attention scores are assigned due to high mutual correlations between materials, and may decrease a dimension of the ANN hidden layer by 1.

According to one embodiment of the present invention, an apparatus and method of predicting a recipe property reflecting similarity between chemical materials are provided to define similarity between rubber materials that may not be considered in a conventional embedding method, and to reconstruct input data by expressing a material as a vector based on the similarity.

In addition, according to the present invention, an accurate property of a rubber recipe is predicted using reconstructed data as input data to an artificial neural network.

The recipe property prediction apparatus 100 may include a material embedding process and an ANN prediction model.

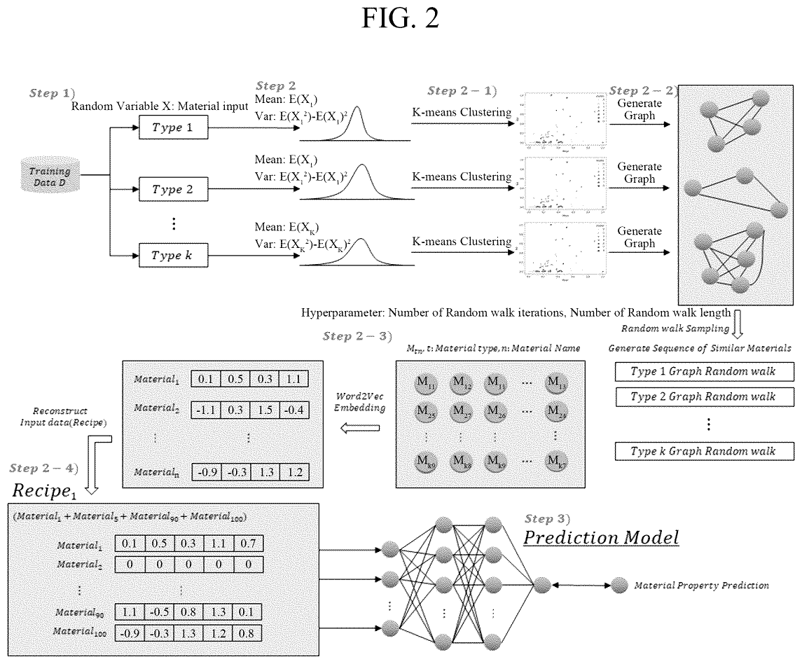

FIG. 2 is a diagram illustrating a whole process of material embedding according to the present invention.

As shown in FIG. 2, in step 1, the recipe property prediction apparatus 100 may distinguish rubber materials, which are training data, by type and may divide them into clusters. The recipe property prediction apparatus 100 may use divided clusters as material inputs using a random variable X.

In step 1, the recipe property prediction apparatus 100 may divide materials, such as rubber, carbon, oil, and the like, into clusters.

In step 2, the recipe property prediction apparatus 100 may calculate a mean E(X₁) and a variance (Var) E(X₁²)−E(X₁)² for the material input.

The recipe property prediction apparatus 100 may again divide the materials already divided into clusters based on an input amount. In this case, the random variable may be an input amount of the material, the x-axis may be a primary moment (mean) of the material, and the y-axis may be a secondary moment (variance) of the material.

In step 2-1, the recipe property prediction apparatus 100 may show a distribution status by performing K-means clustering using the calculated mean and variance.

The recipe property prediction apparatus 100 may obtain a final cluster that reflects the similarity between materials using K-means clustering.

In step 2-2, the recipe property prediction apparatus 100 may generate graph data with the shown distribution status.

The recipe property prediction apparatus 100 may perform random walk sampling on the generated graph data and may generate a sequence of similar materials.

The recipe property prediction apparatus 100 may form graph data with similar materials and may sample a similar material pair through random walk sampling. In this case, the graph data may be constructed as a fully connected graph because similar materials are defined and a user may arbitrarily determine the length and times of random walks.

In step 2-3, the recipe property prediction apparatus 100 may construct a matrix with respect to a material type and material name with the sequence of similar materials.

In addition, the recipe property prediction apparatus 100 may perform Word2Vec embedding on the constructed matrix.

In addition, the recipe property prediction apparatus 100 may reconstruct the matrix on which Word2Vec embedding is performed based on similarity.

The recipe property prediction apparatus 100 may define a random walk sampling result as a sequence and may express materials in the sequence by vectors.

In step 2-4, the recipe property prediction apparatus 100 may output a recipe by selecting significant data in the reconstructed matrix.

The recipe property prediction apparatus 100 may concatenate materials expressed by vectors and may express the materials by a recipe. In this case, a vector of a material that is not an input may be expressed by a zero vector to express the material in a fixed size.

In step 3, the recipe property prediction apparatus 100 may perform material property prediction by inputting the output recipe to the prediction model.

The recipe property prediction apparatus 100 may predict a property by using an input to an ANN.

In the material embedding process, since information on similarity between materials as shown in FIG. 2 is not available, similarity based on data may not be defined.

The type of materials may be divided into rubber R, carbon C, oil O, and the like, and the recipe property prediction apparatus 100 may classify even the same material into different clusters (or types) based on an input amount.

A criterion for clustering based on an input amount may use the primary moment and the secondary moment, which are characteristics of the probability distribution.

It may be supposed that materials in the same cluster have similar properties.

The recipe property prediction apparatus 100 may embed a material using a defined material similarity assumption and may reconstruct a recipe vector using input materials.

The recipe property prediction apparatus 100 may predict a property using the reconstructed recipe vector as an input of the ANN.

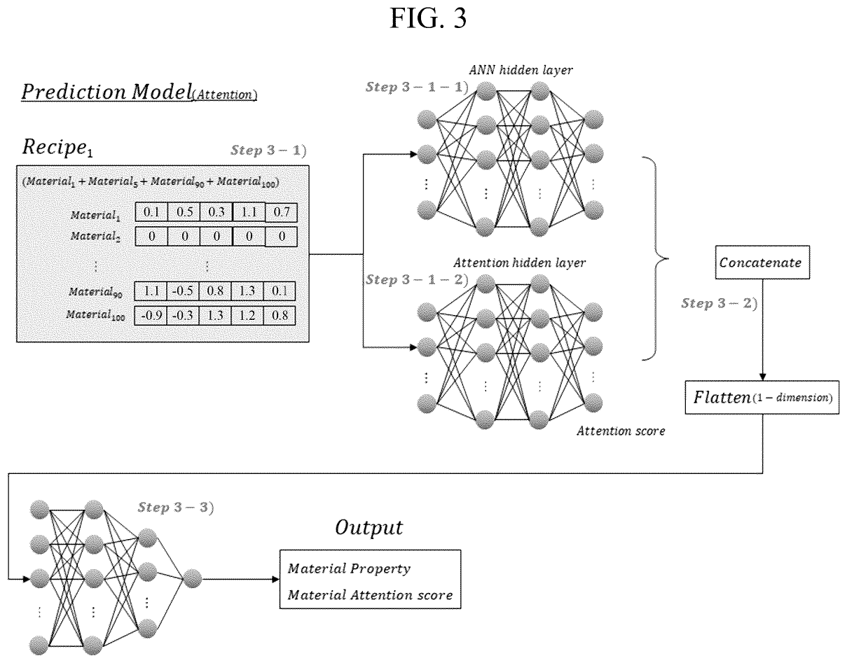

FIG. 3 is a diagram illustrating a property prediction model based on an artificial neural network according to the present invention.

In step 3-1, the recipe property prediction apparatus 100 may apply generated recipe data to a prediction model.

The recipe property prediction apparatus 100 may use a rubber material as an input to an ANN. In this case, the recipe property prediction apparatus 100 may include two hidden layers, which are an ANN hidden layer (step 3-1-1) for linear conversion of a material vector and an attention hidden layer (step 3-1-2) for calculating an attention score of the material vector.

In step 3-1-1 and step 3-1-2, the recipe property prediction apparatus 100 may generate a graph data ANN hidden layer using recipe data and may derive an attention score for each graph data through the prediction model.

In step 3-2, the recipe property prediction apparatus 100 may concatenate the generated graph data by considering the attention score. In addition, the recipe property prediction apparatus 100 may decrease a dimension (1-dimension) by flattening.

The recipe property prediction apparatus 100 may transform a dimension into a one-dimensional vector value after concatenating linearly converted hidden layers.

In step 3-3, the recipe property prediction apparatus 100 may output a material property and a material attention score as a result.

The recipe property prediction apparatus 100 may predict a final property using a result of a previous step as an input to the hidden layer.

In this case, the prediction result may derive two result values, which are a property value and an attention score of the material.

FIG. 3 illustrates an example of an ANN-based property prediction model and describes a detailed structure of an ANN model.

The recipe property prediction apparatus 100 may predict a final property by converting an input material into a vector and using the vector together with an ANN layer that considers the importance of each material.

The importance may be defined as an attention score and the recipe property prediction apparatus 100 may increase a weight as the importance of a material increases.

Such a model may have an advantage of being able to interpret an influence of the material on a property value as well as a prediction value.

According to the present invention, when conducting performance comparison validation with an existing proposed model through K-fold cross-validation in an experiment for predicting six different properties of a rubber product, the performance may increase up to 37% on average compared to existing Word2Vec.

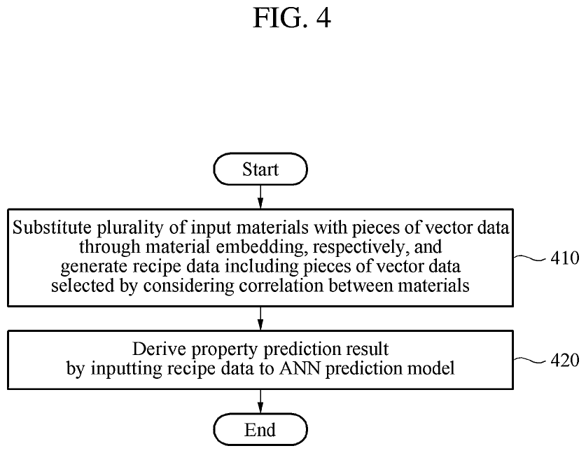

Hereinafter, FIG. 4 describes a workflow of the recipe property prediction apparatus 100 reflecting similarity between chemical materials according to embodiments of the present invention.

FIG. 4 is a flowchart illustrating a method of predicting a recipe property reflecting similarity between chemical materials according to one embodiment of the present invention.

The method of predicting a recipe property reflecting similarity between chemical materials according to one embodiment may be performed by the recipe property prediction apparatus 100 reflecting similarity between chemical materials.

First of all, in operation 110, the recipe property prediction apparatus 100 may substitute a plurality of input materials with pieces of vector data, respectively, through material embedding and may generate recipe data including vector data selected by considering the correlation between materials. Operation 110 may be a process of converting each of input materials, which are substances, into vector data, which is a numerical value, and generating recipe data by selecting and including pieces of vector data having mutual correlation.

In this case, the recipe data may refer to data related to a method of determining a type, an input amount, and the like of a material, which is a raw material for producing a product. The recipe data may function as source data that informs of a property of a product produced by the recipe data based on training thereafter.

The recipe property prediction apparatus 100 may generate recipe data related to product production using vector data of materials having correlations therebetween.

In substituting each input material into vector data, the recipe property prediction apparatus 100 may divide and cluster the plurality of materials by type. That is, the recipe property prediction apparatus 100 may group and divide input materials into clusters by type and category. For example, the recipe property prediction apparatus 100 may divide input materials into clusters, which are rubber R, carbon C, and oil O.

In addition, the recipe property prediction apparatus 100 may substitute divided clusters with vector data using a random variable X.

The random variable may be a function that determines real values as a distribution by an intuitively determined probability with respect to an unknown sample that is a subject of a single observation, and may refer to a function X that assigns a real number x(=X(w)) to an element w in a sample space.

In addition, the processing unit may calculate a mean and a variance (Var) for the substituted vector data. That is, the recipe property prediction apparatus 100 may calculate Mean:E(X₁) and Var:E(X₁²)−E(X₁)² by substituting vector data for a mean calculation equation and variance calculation equation.

In substituting with vector data, the recipe property prediction apparatus 100 may substitute a material, which is predefined but is not an input, with vector data having a determined vector value. That is, the recipe property prediction apparatus 100 may substitute data that is not actually input with vector data of a zero vector.

In the above-described example in which rubber R, carbon C, and oil O are input, when a material, which is predefined but is not an input, is silicon, the recipe property prediction apparatus 100 may substitute the silicon with vector data having a value of “0”.

After calculating the mean and variance, the recipe property prediction apparatus 100 may show a distribution status by performing K-means clustering using the calculated mean and variance.

K-means clustering may be an algorithm for clustering given data into k clusters and may operate in a way that minimizes a variance of a distance difference of each cluster. K-means clustering may iteratively update a center point using the mean coordinates of points allocated to each cluster and may show a distribution status on a virtual plane.

The recipe property prediction apparatus 100 may dispose k centroids on a virtual plane through K-means clustering, may form a cluster by allocating each piece of data to the nearest centroid, may calculate a new centroid based on the allocated cluster, and may show the distribution status by repeating the above-described processes.

In addition, the recipe property prediction apparatus 100 may implement the shown distribution status in graph data. That is, the recipe property prediction apparatus 100 may implement graph data by setting a cluster to be a vertex in the shown distribution status and connecting the clusters with a line.

In addition, the recipe property prediction apparatus 100 may perform random walk sampling on the implemented graph data and may generate a sequence of similar materials. That is, the recipe property prediction apparatus 100 may connect multiple pieces of graph data and may generate a sequence of similar materials by arranging materials based on similarity.

Thereafter, the recipe property prediction apparatus 100 may construct a matrix with respect to a material type and material name with the sequence of similar materials. That is, the recipe property prediction apparatus 100 may express multiple pieces of the connected graph data by a matrix of a material type and material name.

In addition, the recipe property prediction apparatus 100 may reconstruct the matrix by performing Word2Vec embedding. That is, the recipe property prediction apparatus 100 may reconstruct the constructed matrix by converting the matrix into a vector through Word2Vec embedding.

Thereafter, the recipe property prediction apparatus 100 may generate the recipe data by selecting significant data in the reconstructed matrix based on the correlation between the materials. That is, the recipe property prediction apparatus 100 may select significant data having high mutual correlation in the reconstructed matrix and may generate the recipe data by including the selected significant data.

In addition, in operation 420, the recipe property prediction apparatus 100 may input the recipe data into an ANN prediction model and may derive a property prediction result on the recipe data from the ANN prediction model. That is, for recipe data generated by considering the mutual correlation between materials, operation 420 may be a process of predicting a property of a product to be produced based on the recipe data through training.

The recipe property prediction apparatus 100 may apply the recipe data to a prediction model, may form an ANN hidden layer using the recipe data through the prediction model, and may derive the property prediction result from a material property and material attention score using the ANN hidden layer.

That is, the recipe property prediction apparatus 100 may receive an output of a material property and a material attention score as a training result of the prediction model related to the recipe data.

According to one embodiment, the recipe property prediction apparatus 100 may decrease a training processing amount in the prediction model by decreasing a dimension of the ANN hidden layer.

For this, the recipe property prediction apparatus 100 may derive an attention score for each ANN hidden layer, may concatenate the ANN hidden layers by considering the attention scores, and may derive the property prediction result for the ANN hidden layer of which a dimension decreases through flattening.

That is, the recipe property prediction apparatus 100 may sequentially concatenate and flatten ANN hidden layers to which high attention scores are assigned due to high mutual correlations between materials, and may decrease a dimension of the ANN hidden layer by 1.

According to one embodiment of the present invention, an apparatus and method of predicting a recipe property reflecting similarity between chemical materials are provided to define similarity between rubber materials that may not be considered in a conventional embedding method, and to reconstruct input data by expressing a material as a vector based on the similarity.

In addition, according to the present invention, an accurate property of a rubber recipe is predicted using reconstructed data as input data to an ANN.

The methods according to the above-described examples may be recorded in non-transitory computer-readable media including program instructions to implement various operations of the above-described examples. The media may also include, alone or in combination with the program instructions, data files, data structures, and the like. The program instructions recorded on the media may be those specially designed and constructed for the purposes of example embodiments, or they may be of the kind well-known and available to those having skill in the computer software arts. Examples of non-transitory computer-readable media include magnetic media such as hard disks, floppy disks, and magnetic tape, optical media such as CD-ROM discs, DVDs, and/or Blue-ray discs, magneto-optical media such as optical discs, and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter. The above-described devices may be configured to act as one or more software modules in order to perform the operations of the above-described example embodiments, or vice versa.

The software may include a computer program, a piece of code, an instruction, or some combination thereof, to independently or uniformly instruct or configure the processing device to operate as desired. Software and data may be embodied permanently or temporarily in any type of machine, component, physical or pseudo equipment, computer storage medium or device, or in a propagated signal wave capable of providing instructions or data to or being interpreted by the processing device. The software also may be distributed over network-coupled computer systems so that the software is stored and executed in a distributed fashion. The software and data may be stored by one or more non-transitory computer-readable recording mediums.

A number of example embodiments have been described above. Nevertheless, it should be understood that various modifications may be made to these example embodiments. For example, suitable results may be achieved if the described techniques are performed in a different order and/or if components in a described system, architecture, device, or circuit are combined in a different manner and/or replaced or supplemented by other components or their equivalents.

What is claimed is:

1. A method of predicting a recipe property reflecting similarity between chemical materials, the method comprising:
substituting a plurality of input materials with vector data, respectively, through material embedding and generating recipe data comprising pieces of vector data selected by considering a correlation between materials; and
inputting the recipe data to an artificial neural network (ANN) prediction model and deriving a property prediction result on the recipe data from the ANN prediction model.

2. The method of claim 1, wherein the generating of the recipe data comprises:
distinguishing the plurality of materials by type and dividing the plurality of materials into clusters;
substituting each of the divided clusters with vector data using a random variable X; and
calculating a mean and a variance for the vector data.

3. The method of claim 2, wherein the generating of the recipe data further comprises:
for a material, which is predefined but is not an input, substituting with vector data having a determined vector value.

4. The method of claim 2, wherein the generating of the recipe data further comprises:
showing a distribution status by performing K-means clustering using the calculated mean and variance;
implementing the shown distribution status in graph data; and
performing random walk sampling on the implemented graph data and generating a sequence of similar materials.

5. The method of claim 4, wherein the generating of the recipe data further comprises:
constructing a matrix with respect to a material type and material name with the sequence of similar materials;
reconstructing the matrix by performing Word2Vec embedding on the matrix; and
generating the recipe data by selecting significant data in the reconstructed matrix based on a correlation between the materials.

6. The method of claim 1, wherein the deriving of the property prediction result comprises:
applying the recipe data to a prediction model;
forming an ANN hidden layer using the recipe data through the prediction model; and
deriving a material property and a material attention score as the property prediction result using the ANN hidden layer.

7. The method of claim 6, wherein the deriving of the property prediction result further comprises:
deriving attention scores for the ANN hidden layers, respectively; and
concatenating the ANN hidden layers by considering the attention scores and deriving the property prediction result for an ANN hidden layer of which a dimension is decreased by flattening.

8. An apparatus for predicting a recipe property reflecting similarity between chemical materials, the apparatus comprising:
a generation unit configured to substitute a plurality of input materials with pieces of vector data through material embedding, respectively, and generate recipe data comprising pieces of vector data selected by considering a correlation between materials; and
a processing unit configured to input the recipe data to an artificial neural network (ANN) prediction model and derive a property prediction result for the recipe data from the ANN prediction model.

9. The apparatus of claim 8, wherein the generation unit is further configured to:
distinguish the plurality of materials by type and divide the plurality of materials into clusters,
substitute each of the divided clusters with vector data using a random variable X, and
calculate a mean and a variance for the vector data.

10. The apparatus of claim 9, wherein the generation unit is further configured to:
for a material, which is predefined but is not an input, substitute with vector data having a determined vector value.

11. The apparatus of claim 8, wherein the generation unit is further configured to:
show a distribution status by performing K-means clustering using the calculated mean and variance,
implement the shown distribution status in graph data, and
perform random walk sampling on the implemented graph data and generate a sequence of similar materials.

12. The apparatus of claim 11, wherein the generation unit is further configured to:
construct a matrix with respect to a material type and material name with the sequence of similar materials,
reconstruct the matrix by performing Word2Vec embedding on the matrix, and
generate the recipe data by selecting significant data in the reconstructed matrix based on a correlation between the materials.

13. The apparatus of claim 8, wherein the processing unit is further configured to:
apply the recipe data to a prediction model,
form an ANN hidden layer using the recipe data through the prediction model, and
derive a material property and a material attention score as the property prediction result using the ANN hidden layer.

14. The apparatus of claim 13, wherein the processing unit is further configured to:
derive attention scores for the ANN hidden layers, respectively, and
concatenate the ANN hidden layers by considering the attention scores and derive the property prediction result for an ANN hidden layer of which a dimension is decreased by flattening.

15. A non-transitory computer-readable storage medium storing instructions that, when executed by a processor, cause the processor to perform the method of claim 8.
